(19) United States
(12) Patent Application Publication
(10) Pub. No.: US 2022/0273570 A1
(43) Pub. Date: Sep. 1, 2022

(54) Self-Microemulsifying Multi-Deliverable Systems

(71) Applicant: Quicksilver Scientific, Inc., Louisville, CO (US)

(72) Inventors: Christopher W. Shade, Louisville, CO (US); Steven Tieu, Louisville, CO (US); George Gordon Cantlay, IV, Louisville, CO (US)

(73) Assignee: Quicksilver Scientific, Inc., Louisville, CO (US)

(21) Appl. No.: 17/707,067

(22) Filed: Mar. 29, 2022

Related U.S. Application Data
(63) Continuation of application No. PCT/US20/56213, filed on Oct. 18, 2020.
(60) Provisional application No. 62/923,028, filed on Oct. 18, 2019.

(51) Int. Cl. A61K 9/127; A61K 47/24; A61K 47/10; A61K 47/44; A61K 47/14; A61K 31/12; A61K 36/324; A61K 31/352; A61K 31/4375; A61K 31/366; A61K 36/19; A61K 31/05; A61K 31/404; A61K 31/015; A61K 36/185; A61K 33/30; A61K 33/06; A61K 9/00

(52) U.S. Cl. CPC A61K 9/1277; A61K 47/24; A61K 47/10; A61K 47/44; A61K 47/14; A61K 31/12; A61K 36/324; A61K 31/352; A61K 31/4375; A61K 31/366; A61K 36/19; A61K 31/05; A61K 31/404; A61K 31/015; A61K 36/185; A61K 33/30; A61K 33/06; A61K 9/0053

(57) ABSTRACT

Compositions in the form of oil-based solutions that form oil-in-water (OIW) microemulsions in the aqueous environment of the GI tract when taken orally via a hard or soft capsule are described. The resulting microemulsions that form from the oil-based solution within the GI tract can rapidly deliver oil-soluble species and alcohol-soluble species (including PEG derivative assisted alcohol-soluble species) deliverables to the bloodstream through the tissues forming the GI tract. The in situ formed microemulsions resulting from consumption of the encapsulated oil-based solutions include oil-phase microemulsion droplets of monolayer surfactant bound particles suspended in the aqueous continuous phase of the GI tract. The oil-based solutions also may in situ form water-core liposomes suspended in the aqueous continuous phase of the GI tract.

### REFERENCE TO RELATED APPLICATIONS

This application is a continuation of International Application No. PCT/US2020/056213, entitled “Self-Microemulsifying Multi-Deliverable Systems”, filed Oct. 18, 2020, which claims the benefit of U.S. Provisional Application No. 62/923,028 entitled “Self-Emulsifying Delivery Systems” filed Oct. 18, 2019, both of which are incorporated by reference in the entirety.

### BACKGROUND

Nutritional supplements are conventionally introduced to the bloodstream in multiple ways. Supplements taken orally are absorbed at different rates due to different factors. For example, on average about 10% to 20% of a solid supplement taken orally is absorbed. This can be increased to about 30% with an orally taken gel capsule, to about 45% with a transdermal patch, and to about 50% with conventional intra-oral (sublingual) administration. Injections provide from approximately 90% to 100% adsorption into the bloodstream but are uncommonly used for nutritional supplements.

Emulsions are mixtures of two or more liquids that do not solubilize. Thus, the two or more liquids do not form a solution and an identifiable interface exists between the combined liquids. Emulsions may be macroemulsions, pseudo-emulsions, nanoemulsions, or microemulsions. Emulsions may be used for parenteral delivery, ocular delivery, transdermal delivery, oral delivery, and the like. Emulsions include particles in a continuous (carrier) phase. The particles of the emulsion may be liposomal (bilayer), micellular (monolayer), or monolayer surfactant bound, where the oil of the particle is associated with a surfactant system, but an oil core is not necessarily formed.

FIG. 1A and FIG. 1B represent a liposome 100 having a double wall (bilayer) of phospholipids formed from a hydrophilic exterior wall 120 and a hydrophilic interior wall 125. An interior of the double wall 110 is hydrophobic. The hydrophilic interior wall 125 forms a capsule interior 130, to form what may be referred to as a “water-core” liposome. Liposomes may be thought of as small, fluid-filled capsules where the wall of the capsule is formed from two layers of a phospholipid, thus having a bilayer wall. As phospholipids make up the outer membranes of living cells, the liposome 100 also may be thought of as having a permeable membrane wall like a cell, but without a nucleus or the other components of a living cell within the capsule interior 130. The outer and inner walls 120, 125 of the represented liposome 100 are polar and thus water-soluble, while the interior of the bilayer wall 110 is nonpolar and thus oil-soluble.

The described liposome 100 exists in a hydrophilic continuous phase and carries a hydrophilic liquid inside the capsule interior 130 formed by the bilayer membrane. A comparatively minor amount of hydrophobic liquid may be carried in the interior of the double wall 110 of the liposome 100. A common phospholipid used to form liposomes is phosphatidylcholine (PC), a material found in lecithin. In a hydrophobic (nonpolar) continuous phase, the reverse can occur resulting in a bilayer structure having a hydrophobic or “oil-core”. Liposomes such as this may be referred to as “reverse liposomes” as they have “oil” as opposed to “water” cores.

When introduced to the body, liposomes are known to deliver their internal contents to living cells through one of four methods: adsorption, endocytosis, lipid exchange, and fusion. In adsorption, the outer wall of the liposome sticks to the living cell and releases its contents through the outer wall of the living cell into the living cell. In endocytosis, the living cell consumes the liposome, thus bringing the entire liposome into the cell. The cell then dissolves the outer wall of the liposome and releases the liposome contents into the interior of the living cell. In lipid exchange, the liposome opens near the living cell and the living cell takes in the localized high concentration of liposome interior. In fusion, the outer wall of the liposome becomes part of the outer wall of the living cell, thus carrying the contents of the liposome into the enlarged living cell. These pathways allow for a potential 100% transfer of the interior contents of the liposome to the interior of the living cell, if the liposome can be brought sufficiently near the cell and is properly constructed to interact with the outer wall of the living cell.

FIG. 2 represents a flattened side view of the double wall (bilayer) of phospholipids that forms the liposome. The phospholipids have polar, hydrophilic “heads” and less polar, relatively hydrophobic “tails”. In this representation, the heads form the top and bottom of the bilayer, with the tails forming the interior middle. As previously mentioned, some oil-soluble material can reside between the top and bottom layers within the interior area occupied by the tails.

FIG. 3 represents a micelle 300 having a single wall of phospholipids (monolayer) forming a hydrophilic exterior 320 and a hydrophobic interior 310, thus lacking the hydrophilic capsule interior of a liposome. Thus, in relation to a liposome, a micelle lacks a bilayer and does not provide a capsule interior that can contain a water-soluble, hydrophilic core composition. The tails of the phospholipids in the interior of the micelle 300 are closely spaced, potentially compressed into each other. Thus, any hydrophobic composition held within the micelle 400 exists interspersed in the tails of the phospholipid. This is comparable to the situation where oil-soluble compounds reside between the inner and outer layers that form the bilayer of a liposome. The micelle 400 may be thought of as the outer wall of a liposome without the inner wall providing a hydrophilic capsule interior. A polyethylene glycol modified vitamin E, such as tocopheryl polyethylene glycol succinate 1000 (TPGS), also may be used to form micelles in water as the TPGS has a water-soluble head and an oil-soluble tail, thus is in this way similar to a phospholipid. In a nonpolar continuous phase, the reverse can occur resulting in a monolayer structure having a hydrophilic interior. Structures such as this may be referred to as a “reverse micelle”.

FIG. 4 represents a monolayer surfactant bound particle 400 where an associated oil component 450 is associated with the hydrophobic tails 460 of a surfactant. In this representation, the surfactant has formed a circular shape, thus encircling the associated oil component 450 and thus approximating a relatively large, expanded micelle having a distinct oil core, but such encircling is not required for the associated oil component 450 to associate with the hydrophobic tails 460 of the surfactant. When the monolayer surfactant encircles the oil, thus forming the oil core, the resulting surfactant and oil droplet is considered the suspended particle component of a microemulsion, if formed without pressure or shear and is thus thermodynamically stable. In a polar continuous phase, hydrophilic, polar heads 470 of the surfactant will form a hydrophilic exterior and a hydrophobic interior, thus forming a hydrophobic, oil-soluble core. In a hydrophobic (nonpolar) continuous phase, the reverse can occur resulting in a monolayer structure having a hydrophilic (polar) water core. Structures such as this may be referred to as “invert emulsions” or “invert microemulsions”.

Conventional self-emulsifying and self-microemulsifying delivery systems (SEDS or SMEDS) are mixtures of oil, surfactant, co-surfactant, and the desired deliverable. The SMEDS constituents are encapsulated in a hard or soft gel capsule and swallowed. When released into the aqueous gastrointestinal fluid of the stomach, the capsule dissolves to release the constituents of the SMEDS. The constituents are then believed to in situ form the droplets (surfactant and oil particles) of a microemulsion having the aqueous gastrointestinal fluid of the gastrointestinal (GI) tract as the continuous or carrier phase. Thus, upon dissolution, the capsule is believed to form an oil-in-water (OIW) microemulsion in the aqueous environment of the stomach.

If the OIW microemulsion is formed in the stomach, an oil-soluble species deliverable is carried in the oil of the surfactant and oil particles, permitting delivery of the oil-soluble species deliverable to the tissues of the GI tract. Thus, the system is intended to increase transfer of the oil-soluble species deliverable to the bloodstream, even though the deliverable is substantially not soluble in the aqueous stomach environment.

Curcuminoids are extracted from turmeric power, a product of the Curcuma longa plant. There are three curcuminoids: curcumin, methoxycurcumin, and bisdemethoxycurcumin. Curcumin is a solid, alcohol-soluble species that is insoluble in water, and thus has extremely poor absorption into the GI tract. The solubility of solid curcumin is reported to be 11 ng/mL in water buffered to pH 5.0, and the subsequent bioavailability of orally administered solid curcumin powder in rats is reported to be 1%. Thus, for solid powders, quantifiable serum levels in humans were not reported to be achieved until a large oral dose of 3.6 grams of the solid powder was consumed. It follows that an individual likely would have to consume at least 3.6 grams of curcumin powder per day to have a desired pharmacological effect.

When orally consumed, free curcumin is known to be present at extremely low levels in the body, while curcumin metabolites (curcumin-glucuronide and curcumin-sulfate) are primarily found due to the free curcumin being conjugated when absorbed through the intestine, with curcumin-glucuronide being the primary of the two metabolites. However, it is reported that the effects of curcumin-glucuronide are weaker than those of free curcumin on gene expression in the human hepatoma cell line (HepG2).

Curcumin is reported to have desirable biological activities including anti-inflammatory, anti-viral, anti-cancer, antioxidant, and anti-depression ability. Curcumin also is reported to potentially have the unusual ability as a non-cannabinoid to modulate CB1 and CB2 receptors, as do the cannabinoids. A readily available source of curcumin powder is the CURCUMIN C3 COMPLEX™, as available from Sabinsa Corporation, East Windsor, N.J.; however, other sources of curcumin are available.

Beta caryophyllene is a liquid, oil-soluble species that is insoluble in water and known to have low bioavailability in aqueous systems, such as the GI tract. Beta caryophyllene is a terpene (bicyclic sesquiterpene) found in the essential oils of plants including clove, hops, black pepper, rosemary, and cannabis. It is attributed with anti-inflammatory properties, the unusual ability as a non-cannabinoid to activate the endocannabinoid CB2 receptors, providing an increase in mitochondrial function, and potentially reducing neurodegeneration. Beta caryophyllene may provide the inflammation reduction activity associated with cannabidiol (CBD), but have a stronger pain reduction activity.

Boswellia serrata (frankincense) is a solid, alcohol-soluble species that is insoluble in water and known to have extremely low bioavailability in aqueous systems, such as the GI tract. Boswellia serrata is a polyphenol (pentacyclic triterpene) extract from the Boswellia serrata tree and is reported to reduce inflammation due to blocking leukotriene function, especially in the context of joint and bowel inflammation. The extract includes boswellic acids to which the biological activity is attributed.

Quercetin is a solid, alcohol-soluble species that is insoluble in water and known to have extremely low bioavailability in aqueous systems, such as the GI tract. Quercetin is a plant-derived polyphenol from the flavonoid group, and is found in many fruits, vegetables, leaves, and grains. When IV administered, quercetin acts as an antioxidant by scavenging (deactivating) free radicals, such as oxygen radicals, and as an activator of estrogen receptors. However, the bioavailability of quercetin in humans is low and highly variable (0-50%), and quercetin is rapidly cleared from the body with an elimination half-life of 1-2 hours after oral ingestion of quercetin containing foods or supplements. Following dietary ingestion, quercetin undergoes rapid and extensive metabolism that makes the biological effects observed in IV administered studies unlikely to apply to conventional oral administration.

Berberine HCl is a solid, alcohol-soluble species that is slightly soluble in water and known to have low bioavailability in aqueous systems, such as the GI tract. Berberine is an acid salt (isoquinoline alkaloid) from the protoberberine group of benzylisoquinoline alkaloids found in such plants as berberis, goldenseal, goldthread, tree turmeric, and others. The extract is believed to provide a decrease in insulin resistance, promote glycolysis, and otherwise provide a benefit in the context of blood sugar control.

Milk thistle extract is a solid, alcohol-soluble species that is insoluble in water and known to have low bioavailability in aqueous systems, such as the GI tract. Milk thistle extract is reported to have an antioxidant effect attributed to the flavonoid silymarin and to assist in removing heavy metals, alcohol, and pesticides from the liver. The antioxidant effect is believed to protect the outside of cells against oxidative damage and the resulting potential cell mutation that can occur from the oxidative damage, in addition to increasing liver glutathione levels. Milk thistle extract is an extract of the milk thistle plant including at least 50% silymarin by weight, and preferably at least 70% silymarin by weight. Silymarin, the attributed primary active ingredient in milk thistle extract, is a solid in pure form and stated to have a 20% to at most 50% absorption when milk thistle extract is consumed orally.

Artemisinin is a solid, alcohol-soluble species having poor solubility in both oils and water and is associated with poor bioavailability unless chemically modified. Artemisinin is a sesquiterpene lactone containing a peroxide bridge and is derived from the Asian plant Artemisia annua. Artemisinin is reported to have anti-parasitic and anti-malarial activity and potential anti-cancer and anti-viral activity.

Andrographis is a solid, alcohol-soluble species with poor solubility in water. Andrographis is a diterpene lactone reported to have anti-microbial, anti-inflammatory, and antioxidant activity.

Luteolin is a solid, alcohol-soluble species that is slightly soluble in water. Luteolin is a flavone found in celery, broccoli, green pepper, parsley, thyme, dandelion, perilla, chamomile tea, carrots, olive oil, peppermint, rosemary, navel oranges, and oregano. Plants rich in luteolin have been used in Chinese traditional medicine for treating various diseases such as hypertension, inflammatory disorders, and cancer.

Resveratrol is a solid, alcohol-soluble species that is insoluble in water. Resveratrol (3,5,4′-trihydroxy-trans-stilbene) is a polyphenol produced by several plants in response to injury or, when the plant is under attack by pathogens, such as bacteria or fungi. Sources of resveratrol in food include the skin of grapes, blueberries, raspberries, and mulberries. Resveratrol exists as two geometric isomers: cis-(Z) and trans-(E). The trans- and cis-resveratrol isomers can be either free or bound to glucose. While 70% of orally administered resveratrol is absorbed by the body, the bioavailability is only about 0.5% as resveratrol is extensively metabolized into its glucuronide conjugate by the liver and intestine before reaching the bloodstream. Due to the poor bioavailability, animal models suggest that from 200 to 500 milligrams (mg) of resveratrol is needed per kilogram of diet to affect cell-signaling pathways important for mitochondrial biogenesis.

Diindolylmethane (DIM) is a solid, alcohol-soluble species that is insoluble in water. DIM is an amine derived from indole-3-carbinol which is found in cruciferous vegetables such as broccoli, brussels sprouts, cabbage, and kale. DIM is reported to induce the antioxidant response element (ARE).

Hesperetin is a solid, alcohol-soluble species that is insoluble in water. Hesperetin is a polyphenol flavanone often obtained from citrus fruits and reported as having a positive effect on blood vessel conditions including poor circulation. While the specific mechanism is not presently known, it is believed that Hesperetin may operate by reducing inflammation of the vessels.

Cannabinoids are a class of compounds that act on the cannabinoid receptors in cells that alter neurotransmitter release. Cannabinoids include the endocannabinoids, which are produced naturally in the bodies of animals, phytocannabinoids, which are found in plants of the Cannabis genus and in some other plants, and synthetic cannabinoids that are synthesized. Type 1 cannabinoid receptors are found primarily in the brain and are absent from the part of the brain stem responsible for respiratory and cardiovascular function. Type 2 cannabinoid receptors are predominantly found in the immune system and appear to be responsible for the anti-inflammatory and potentially other therapeutic effects attributed to cannabinoids.

Phytocannabinoids are isolated from plants of the Cannabis genus, which is believed to include three species, Cannabis sativa, cannabis indica, and Cannabis ruderalis. Cannabis plants including less than 0.3% tetrahydrocannabinol (THC) by weight are commonly referred to as “hemp”, while plants including 0.3% or greater by weight THC are commonly referred to as “marijuana”. At least 113 different phytocannabinoids may be isolated from plants of the Cannabis genus. The phytocannabinoids are isolated in their “A” or acidic form and are then decarboxylated, often by heat, to their more biologically active, decarboxylated forms.

THC is the most famous cannabinoid as it binds to the Type 1 receptors and is considered psychoactive. Cannabidiol (CBD) is becoming a more commonly known non-psychoactive cannabinoid as it acts on the Type 1 and Type 2 receptors and is known to reduce pain and inflammation and calm some nerve responses, such as those associated with Dravet syndrome in children. Additionally, CBD may counteract cognitive impairment associated with THC use, including short term memory loss and may have additional anti-psychotic effects in addition to serving as an antioxidant. Cannabigerol (CBG) is another non-psychoactive cannabinoid that may have similar effects to CBD. Cannabichromene (CBC), Cannabinol (CBN), and Cannabitriol (CBT) are other cannabinoids that are being studied for potential biological activity.

Cannabis extracts are oil-soluble species extracted from a plant of the Cannabis genus and are insoluble in water, thus being phytocannabinoids. Preferable cannabis extracts include cannabidiol (CBD), tetrahydrocannabinol (THC), and other cannabinoids including cannabinol (CBN), cannabigerol (CBG), tetrahydrocannabivarin (THCV), cannabidivarin (CBDV), and cannabichromene (CBC). Preferred cannabis extracts include at least 30% by weight CBD and/or THC, while more preferred cannabis extracts include at least 60% by weight CBD and/or THC. Most preferred cannabis extracts include at least 80% by weight CBD and/or THC. “Hemp oil” is a cannabis extract having a concentration of less than 0.3% tetrahydrocannabinol (THC) and a relatively high concentration of cannabidiol (CBD)—thus lacking a psychoactive effect.

Oral delivery of cannabis extracts with conventional delivery systems can result in negligible blood concentration after 20-minutes of administration, and not provide an effective bloodstream concentration, which is believed to be approximately 0.4 ng/mL and higher in the bloodstream. In fact, for low absorbing individuals, effective bloodstream concentrations may not be reached after 90-minutes of oral administration, or at all, with conventional oil delivery systems without consuming undesirably large quantities of the conventional oral delivery system. Hence, of the cannabinoids orally consumed with conventional oil delivery systems, a significant majority of that consumed may be excreted and never used.

While work remains ongoing regarding the health benefits of cannabinoids, pharmacological utility has been demonstrated or is likely to be demonstrated for the previously mentioned Dravet syndrome in children, Parkinson's disease, schizophrenia, anxiety disorder, and inhibiting the development of some cancer cells. As the human endocannabinoid system is involved in basic life functions, including appetite, immune response, reproduction, and pain management, the effects of cannabinoids on the human body are likely diverse. The ability of cannabinoids to prevent the overactivation of these functions may provide a reduction in the progression or prevention of disease based on overactivation of these functions.

Terpenes are liquid, oil-soluble extracts that are insoluble in water. Terpenes may be extracted from plants including conifers, flowers, citrus fruits, and some insects, such as termites and swallowtail butterflies. From a molecular perspective, all terpenes include isoprene functionality and are a diverse class of organic molecules. In addition to historical use as fragrances, terpenes provide a basis for biologically active substances including Vitamin-A, Vitamin-D, and steroids. Terpenes include compounds such as limonene, pinene, linalool, and the previously discussed beta-caryophyllene. Oral delivery of terpenes with conventional delivery systems can result in negligible blood concentrations and fail to provide an effective bloodstream concentration.

Zinc is a solid mineral generally available as a water-soluble salt, such as zinc gluconate, zinc citrate, zinc acetate, zinc picolinate, zinc sulfate, and others. Zinc is considered an essential nutrient and thus the body cannot produce or store it. Zinc has many roles in gene expression, enzymatic reactions, protein and DNA synthesis, and is a key component of a healthy immune system, especially in the context of resisting viral infection.

While each of the forementioned plant extracts have been claimed to have potential health benefits, a problem with obtaining any actual therapeutic benefit from their consumption has been a lack of bioavailability. As an example, for curcumin to show any potentially useful blood concentration, a human must eat over 3 grams a day, and in fact, larger amounts may be required to obtain the necessary therapeutic dose for a health benefit. While 3+ grams of curcumin per day technically may be ingestible, few would consider such a large quantity edible. Furthermore, curcumin is not the most difficult to deliver of these plant extracts. For example, without chemical modification, artemisinin is essentially non-deliverable with conventional delivery systems.

Of these potentially beneficial to human health deliverables, curcumin, for example, is a deliverable where delivery via self-microemulsifying delivery systems (SMEDS) is a goal due to the water insolubility, and thus extremely poor bioavailability, which otherwise requires very high dosages of the orally unpalatable curcumin. Thus, conventional SMEDS have been attempted to deliver curcumin to the bloodstream while bypassing the water insolubility of the curcumin and having to eat/taste multiple grams of raw curcumin on a daily basis.

While conventional SMEDS have been attempted for curcumin, these conventional systems have disadvantages including limited deliverable choice, low solubility of the deliverable in the SMEDS delivery constituents, and inferior emulsion formation. The low solubility of the deliverable in the SMEDS delivery constituents and inferior emulsion formation in combination result in relatively poor delivery of the deliverable to the bloodstream, often not exceeding that of conventional oil delivery methods. Inferior emulsion formation results in unstable and/or relatively large oil-core particle formation above 100 nm in the GI tract. For example, particles having an average diameter of 100 nm are believed to have only an approximately 40% delivery to the bloodstream through the GI tract, while particles having an average diameter of 75 nm are believed to have and approximate 60% delivery through the GI tract. Low solubility of the deliverable in the SMEDS delivery constituents results in relatively little deliverable in any emulsion that is formed in the GI tract. Without the deliverable being part of an in situ formed emulsion, the deliverable is being delivered conventionally, not by a SMEDS.

The self-microemulsifying delivery systems and methods of the present invention overcome at least one of the disadvantages associated with conventional SMEDS.

### SUMMARY

In one aspect, the invention provides a composition for delivering a deliverable to the gastrointestinal tract, the composition including an exterior capsule enclosing an oil-based solution; where the oil-based solution includes an emulsion system and a deliverable, where the emulsion system includes a surfactant system, an emulsion oil system, and a resin system, and where the deliverable is chosen from an oil-soluble species, an alcohol-soluble species, and combinations thereof.

In another aspect of the invention, there is a method of making the composition for delivering a deliverable to the gastrointestinal tract, the method including heating an alcohol and water solution to a low temperature of 65° C. to 78° C., where the alcohol and water solution has an alcohol to water ratio from 80:20 to 97:3 on a volume basis to form a heated solvent solution; combining the alcohol-soluble species deliverables with the heated solvent solution to form a heated deliverable mixture; combining the surfactant system and the resin system with the heated deliverable mixture; increasing the heated deliverable mixture above 78° C. to form a reduced solution; and combining the emulsion oil system with the reduced solution to form the oil-based solution.

In another aspect of the invention, there is an ingestible and edible composition for pain relief, the composition including an encapsulated oil-based solution including from 2 to 3 percent by weight phospholipid, from 24 to 30 percent by weight polyethylene glycol derivative, from 8 to 13 percent by weight turmeric oleoresin, from 1 to 2.5 percent by weight propolis, from 12 to 18 percent by weight emulsion oil, from 23 to 31 percent by weight turmeric oil, from 5 to 9 percent by weight beta caryophyllene, from 1.5 to 4 percent by weight hemp oil, from 1 to 3 percent by weight piperine, from 4 to 4 percent by weight curcumin, and from 2 to 4 percent by weight Boswellia serrata.

In another aspect of the invention, there is an ingestible and edible composition for balancing microbial load in a mammal, the composition including an encapsulated oil-based solution including from 3.2 to 5 percent by weight phospholipid, from 26.3 to 30 percent by weight polyethylene glycol derivative, from 3 to 7 percent by weight turmeric oleoresin, from 2.6 to 4 percent by weight propolis, from 18.2 to 23 percent by weight emulsion oil, from 11 to 20 percent by weight turmeric oil, from 1 to 5 percent by weight cinnamon oil, from 1 to 5 percent by weight peppermint oil, from 0.2 to 1.3 percent by weight hemp oil, from 0.3 to 2 percent by weight berberine HCl, from 2 to 5 percent by weight milk thistle extract, from 3 to 7 percent by weight artemisinin, from 0.3 to 2 percent by weight andrographis, from 2 to 6 percent by weight Boswellia serrata, and from 2 to 4 percent by weight quercetin.

In another aspect of the invention, there is an ingestible and edible composition for controlling inflammation, the composition including an encapsulated oil-based solution including from 1 to 3 percent by weight phospholipid, from 25 to 34 percent by weight polyethylene glycol derivative, from 6 to 10 percent by weight turmeric oleoresin, from 8 to 13 percent by weight associating oil, from 27 to 35 percent by weight turmeric oil, from 2 to 6 percent by weight cinnamon oil, from 7 to 10 percent by weight spearmint oil, from 2 to 5 percent by weight berberine HCl, from 2 to 5 percent by weight milk thistle extract, from 2 to 5 percent by weight resveratrol, from 2 to 5 percent by weight hesperetin, and from 2 to 5 percent by weight quercetin.

In another aspect of the invention, there is an ingestible and edible composition for supplementing dietary zinc in a mammal, the composition including an encapsulated oil-based solution including from 1 to 3 percent by weight phospholipid, from 25 to 34 percent by weight polyethylene glycol derivative, from 7 to 10 percent by weight propolis, from 22 to 30 percent by weight associating oil, from 10 to 15 percent by weight turmeric oil, from 10 to 15 percent by weight spearmint oil, from 3 to 5 percent by weight zinc acetate, from 2 to 5 percent by weight luteolin, from 2 to 5 percent by weight hesperetin, and from 2 to 5 percent by weight quercetin.

The scope of the present invention is defined solely by the appended claims and is not affected by the statements within this summary.

### DESCRIPTION OF THE FIGURES

The invention can be better understood with reference to the following figures and description. The components in the figures are not necessarily to scale, emphasis instead being placed upon illustrating the principles of the invention.

FIG. 1A and FIG. 1B represent a liposome having a double wall (bilayer) of phospholipids formed from a hydrophilic exterior wall and a hydrophilic interior wall.

FIG. 2 represents a flattened side view of the double wall (bilayer) of phospholipids that forms the liposome.

FIG. 3 represents a micelle having a single wall of phospholipids (monolayer) forming a hydrophilic exterior and a hydrophobic interior lacking the hydrophilic capsule interior of a liposome.

FIG. 4 represents a monolayer surfactant bound particle where an associated oil component is associated with the hydrophobic tails of a surfactant.

FIG. 5 represents a method of making the composition including the encapsulated oil-based solution.

FIG. 6 provides the results from a LC/MS/MS plasma curcumin analysis at the 0, 10, 20, 40, 60, 90, 120, 180, and 480 (commercially available only) minute time intervals.

FIG. 7 provides the results from a LC/MS/MS plasma curcumin-glucuronide analysis at the 0, 10, 20, 40, 60, 90, 120, 180, and 480 (commercially available only) minute time intervals.

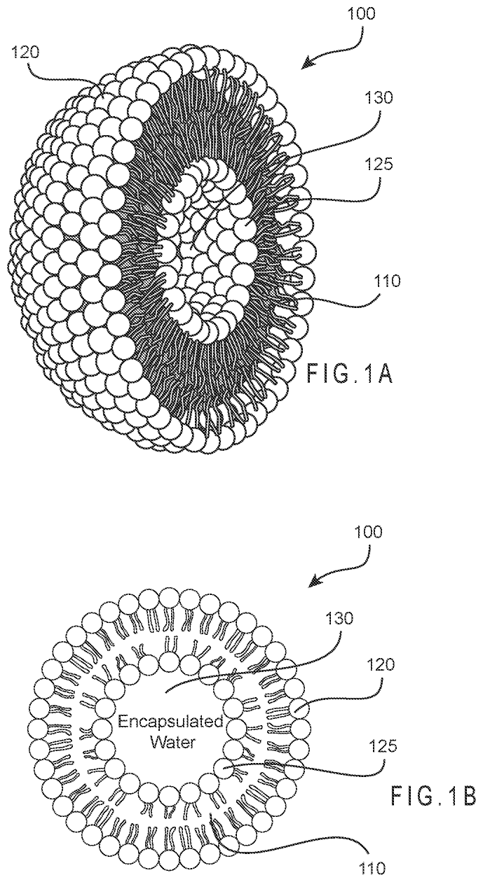

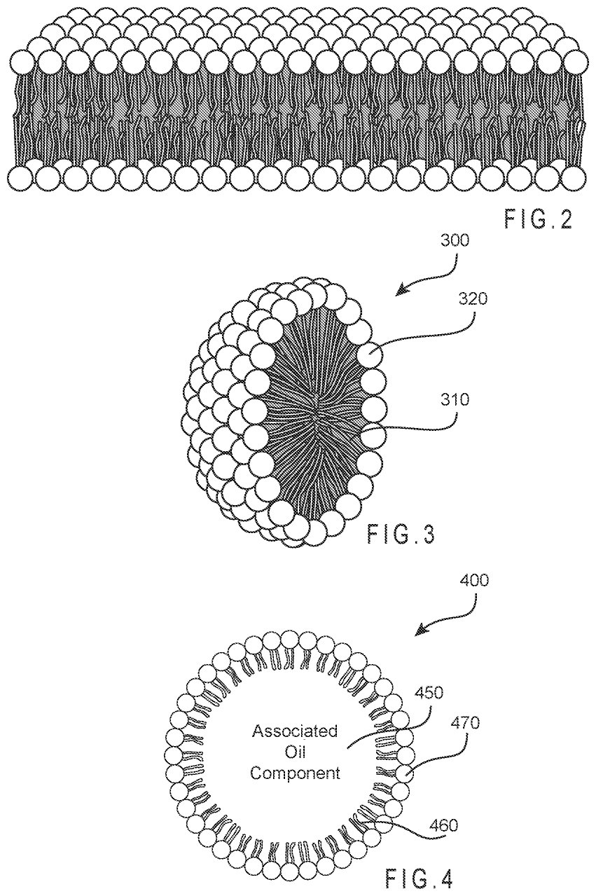

### DETAILED DESCRIPTION

Compositions in the form of oil-based solutions that form oil-in-water (OIW) microemulsions in the aqueous environment of the GI tract when taken orally via a hard or soft capsule are described. The resulting microemulsions that form from the oil-based solution within the GI tract can rapidly deliver oil-soluble species and alcohol-soluble species (including PEG derivative assisted alcohol-soluble species) deliverables to the bloodstream through the tissues forming the GI tract. The in situ formed microemulsions resulting from consumption of the encapsulated oil-based solutions include oil-phase microemulsion droplets of monolayer surfactant bound particles suspended in the aqueous continuous phase of the GI tract. The oil-based solutions also may in situ form water-core liposomes suspended in the aqueous continuous phase of the GI tract.

The exterior capsule containing the oil-based solutions may be of the “hard shell” or “soft shell” variety. Useful hard-shell capsules are made from aqueous solutions of gelling agents, such as animal protein (mainly gelatin) or plant polysaccharides or their derivatives (such as carrageenan and modified forms of starch and cellulose). Other additional ingredients are a gelling agent such as glycerin or sorbitol to decreases the capsule's hardness, coloring agents, preservatives, disintegrates, lubricants and surface treatments. Examples of exterior hard-shell capsules include Nutra Pak capsules as available from NutraPak USA, East Rutherford, N.J. or QUALICAPS™ as available from Qualicaps, Inc., Whitsett, N.C. Useful soft-shell capsules are typically a combination of gelatin, water, opacifier and a plasticizer such as glycerin or sorbitol. The main source of gelatin is collagen, found in the skin and bones of animals, and is typically sourced from bovine or porcine. Vegetarian capsules are mainly made from Hydroxy Propyl Methyl Cellulose (HPMC) and alternatively polyethylene oxides (PEOs). Examples of exterior soft-shell capsules are available from NutraPak USA or from Soft Gel Technologies, Inc., Los Angeles, Calif. In either instance, the hard- or soft-shell capsule material is selected to not adversely affect the encapsulated oil-based solution that is released into the GI tract.

In the oil-based solutions that are encapsulated by the exterior capsule, solid deliverables are solubilized in the liquids of the oil-based solution. The resulting solutions are believed to form microemulsion droplets having an inner oil-based core, and intermediate resinous layer encapsulating the inner core, and an outer surfactant monolayer encapsulating the intermediate layer when placed in aqueous environments, such as the GI tract. While being solubilized in each other, the different solubilities throughout these layered structures are believed to allow for the inclusion of a wide diversity of deliverables, including those having alcohol- and/or oil-solubility. The compositions provide improved deliverable solubility and bloodstream uptake in relation to conventional SMEDS when introduced into the GI tract.

The emulsion system that forms the encapsulated oil-based solution of the SMEDS, includes a surfactant system, an emulsion oil system, and a resin system. The emulsion system may be designed to produce water-core liposomes in addition to microemulsion droplets in an aqueous continuous phase, such as the GI tract. Regardless of the specific constituents used in the emulsion system, one or more deliverables are included and solubilized in the encapsulated oil-based solution. Thus, the surfactant system, the emulsion oil system, the resin system, and the deliverable/s form the oil-based solution. This is believed to contrast with conventional SMEDS systems where the deliverable/s are not fully or poorly solvated in the emulsion forming components or where the conventional emulsion system is water- as opposed to oil-based.

When released into the aqueous environment of the GI tract, the oil-based solution forms microemulsion droplets, and optionally liposomes, that include the deliverables and have an average droplet diameter of 10 to 100 nanometers (nm) and a preferable average droplet diameter of 10 to 80 nm. More preferably, the formed microemulsion droplets and any included liposomes have an average particle diameter of 10 to 60 nm. Thus, these average droplet diameters are observed in an aqueous environment for the formed microemulsion droplets including any deliverables and for any formed liposomes including water-soluble deliverables.

The surfactant system of the emulsion system includes a phospholipid, such as phosphatidylcholine (PC), and a polyethylene glycol derivative, such as tocopheryl polyethylene glycol succinate (TPGS). Additional surfactants may be included in the surfactant system, if the additional surfactants are compatible with formation of the desired monolayer surfactant bound particles forming the microemulsion droplets and the optional water-core liposomes.

The phospholipid of the surfactant system is a glycerophospholipid preferably isolated from lecithin. As the phospholipid is preferably a lecithin isolate, the named isolates preferably include 80% (w/w) of the specified phospholipid with the remaining constituents being one or more additional phospholipids isolated from the lecithin or other lecithin isolates. Preferred phospholipid lecithin isolates include phosphatidylcholine (PC), phosphatidylethanolamine (PE), phosphatidylinositol (PI), ceramide phosphoryl ethanolamine (Cer-PE), ceramide phosphoryl choline (SPH), and combinations thereof, with PC, PE, and combinations thereof being more preferred.

The polyethylene glycol derivative of the surfactant system may be a polyethylene glycol modified vitamin E, such as tocopheryl polyethylene glycol succinate 1000 (TPGS), polysorbate 40, polysorbate 60, or polysorbate 80. Preferably, the polyethylene glycol derivate is TPGS, polysorbate 40, or polysorbate 80. More preferably, the polyethylene glycol derivative is TPGS or polysorbate 40. TPGS, polysorbate 20, polysorbate 40, polysorbate 60, and polysorbate 80 are often thought of as interchangeable. However, polysorbate 20 is less preferred as it is less likely to form the desired microemulsions in combination with the phospholipid.

Preferably, the surfactant system constitutes from 27% to 35% by weight of the oil-based solution including one or more deliverables that forms the OIW microemulsion when released into the GI tract. The phospholipid preferably constitutes from 1% to 5% of the oil-based solution, while the polyethylene glycol derivative preferably constitutes from 26% to 30% of the oil-based solution. Thus, the preferred ratio of phospholipid to polyethylene glycol derivative in the oil-based solution is from 1:5 to 1:30.

The emulsion oil system of the emulsion system includes an associating oil including at least one of medium chain triglycerides (MCT), citrus oil, and combinations thereof. By “associating” it is meant that the oil can be held within the phospholipid/polyethylene glycol derivative monolayer. MCT oils are triglycerides whose fatty acids have an aliphatic tail of 6-12 carbon atoms. Preferable MCT oils include caproic acid (hexanoic acid), caprylic acid (octanoic acid), capric acid (decanoic acid), lauric acid (dodecanoic acid), and combinations thereof. More preferred MCT oils include caprylic acid, capric acid, and combinations thereof. Preferred citrus oils include orange oil, lemon oil, and combinations thereof.

In addition to the associating oil, the emulsion oil system preferably includes a terpene oil, such as turmeric oil, cinnamon oil, peppermint oil, spearmint oil, or a blend of terpene oils.

Preferably, the emulsion oil system constitutes from 38% to 55% by weight of the oil-based solution including one or more deliverables that forms the OIW microemulsion when released into the GI tract. The associating oil preferably constitutes from 8% to 28% of the oil-based solution. When included, the terpene oil or terpene oil blend preferably constitutes from 18% to 46% of the oil-based solution. Thus, the preferred ratio of associating oil to terpene oil or terpene oil blend in the oil-based solution is from 1:1.7 to 1:5.5, when a terpene oil or terpene oil blend is included.

The resin system of the emulsion system includes at least one resin chosen from turmeric oleoresins, propolis, astaxanthin oleoresin, pine resin, ginger oleoresin, and combinations thereof. The resin of the resin system may be a solid or semisolid with some viscous oily fluid content and is insoluble in water. While the resin system may include some oil, there is enough solid component that it would not be considered a liquid, at most being considered to have a tightly bound liquid oil constituent. The resin or resins of the resin system are part of the emulsion system; however, they also may provide biological activity.

Turmeric oleoresins are oil-soluble species and are extracted from the Curcuma longa plant, but in relation to curcuminoids, which are “hard” solids, turmeric oleoresins are thick, waxy pastes that may include an oil component. Turmeric oleoresins include from 37-55% curcuminoids by weight and up to 25% volatile oil by weight. Turmeric oleoresin is isolated as one of the three recovered fractions from processing turmeric rhizomes. As turmeric oleoresin includes curcuminoids, the oleoresin may provide the biological activity of curcumin.

To obtain turmeric oleoresins, generally, turmeric rhizomes are dried and ground into a powder. Then the powder is extracted with solvents (some combination of acetone, dichloromethane, 1,2-dichloromethane, methanol, ethanol, isopropanol, and hexane) to provide a solid, waxy component that is the turmeric oleoresin, turmeric oil, and solvent solubilized solid curcumin. Thus, while the oleoresin includes some oil, it is the oil trapped in or with the solids, as the light or “essential turmeric oil” is removed during extraction processing. The “light” or “essential” recovered turmeric oil may form part of the emulsion oil system of the oil-based solution as previously discussed. The solvent is removed from the extraction solvent solubilized curcumin to produce curcumin powder, as also previously discussed.

Propolis is a resinous substance including polyphenols of the flavonoid class that is produced by honeybees from tree buds and is used by the bees to fill crevices and otherwise seal honeycombs. Propolis generally includes about 50% resin, 30% wax, 10% oil, 10% pollen, and organics. Due to the varied components, propolis may be considered a complex mixture having water-, alcohol-, and oil-soluble constituents when extracted. Preferably, a propolis ethanol extract may be used as the resin “propolis” to form the or a portion of the resin system for the oil-based solution; however, other propolis extracts may be used in the resin system. More preferably, the propolis extract used in the resin system includes at least 50%, most preferably at least 60%, extracted propolis by weight. Propolis is attributed with improving the immune system, reducing inflammation, and promoting better blood circulation, in addition to other benefits.

Astaxanthin oleoresin is a waxy-solid, alcohol-soluble species that is insoluble in water. Astaxanthin oleoresin may be extracted from algae, plants, and animals. Astaxanthin is a terpene known to have antioxidant properties that provides the red color to shrimp, lobster, and salmon, for example. Astaxanthin cannot be synthesized by mammals, and thus must be provided through the diet.

Preferably, the resin system constitutes from 3% to 18% by weight of the oil-based solution including one or more deliverables that forms the OIW microemulsion when released into the GI tract. When used, the turmeric oleoresin preferably constitutes from 3% to 14% of the oil-based solution, while when used, propolis preferably constitutes from 1% to 10% of the oil-based solution. When used in combination, ratios of propolis to turmeric oleoresin from 1:1.7 to 1:5 are preferred.

The ratio of the resin system to the surfactant system to the emulsion oil system is preferably 1:2-4:3.5-6 by weight, with deviations up to 20% by weight being included, and with deviations up to 10% being more preferred, thus 1:2-4:3.5-6±20% by weight or 1:2-4:3.5-6±10% more preferred by weight.

The oil-based solution may optionally include other ingredients or “adjuvants” that are chemically compatible with the oil-soluble species or alcohol-soluble species deliverables and the emulsion system and that do not substantially interfere with microemulsion or liposome formation. Such adjuvants may include preservatives, antioxidants, electrolytes, fillers, and pigments. Other adjuvants may be used.

The deliverable may be in liquid and/or solid form and be an alcohol-soluble species, an oil-soluble species, or water-soluble. Preferably at least one solid deliverable is included. More preferably, the oil-based solution includes at least two deliverables, with at least one alcohol-soluble and at least one oil-soluble being included. A benefit of the emulsion system in relation to conventional SMEDS is the emulsion system's ability to solubilize multiple, different alcohol- and oil-soluble species, thus allowing for substantially simultaneous delivery of both alcohol- and oil-soluble species deliverables to the GI tract. The emulsion system's believed ability to also form water-core liposomes in addition to the OIW microemulsion and thus deliver water-soluble deliverables via liposome simultaneously with the alcohol- and/or oil-soluble species deliverables is additionally beneficial.

Useful alcohol-soluble species deliverables include curcumin, Boswellia serrata, quercetin, berberine HCl, milk thistle extract, artemisinin, andrographis, luteolin, resveratrol, diindolylmethane, and hesperetin. Other alcohol-soluble species may be used with the emulsion system that do not interfere with microemulsion formation and that may be solubilized in the oil-based solution.

Useful oil-soluble species deliverables include beta caryophyllene and cannabis extracts. Other oil-soluble species may be used with the emulsion system that do not interfere with microemulsion formation and that may be solubilized in the oil-based solution.

Useful water-soluble deliverables include mineral salts, such as zinc, magnesium, and calcium salts. Other water-solubles may be used with the emulsion system that do not interfere with microemulsion formation and that may be solubilized in the oil-based solution due to the non-polar character of the counterion associated with the mineral.

One gram of the oil-based solution can solubilize from 50 mg to 200 mg of deliverable, preferably from 100 mg to 200 mg of deliverable, and more preferably from 120 mg to 180 mg of deliverable, based on the specific deliverables selected for inclusion in the oil-based solution that forms the OIW microemulsion when released into the GI tract. Thus, the encapsulated oil-based solution includes from 10% to 20% deliverable by weight, preferably from 12% to 18% delivered by weight. While higher deliverable loading may be used, solubility of the solid phase deliverables is less likely and insolubilized deliverables will have significantly decreased to no bioavailability. Thus, including insolubilized oil- or alcohol-soluble species deliverables in the oil-based solution will provide little to no increase in bioavailability of the insolubilized deliverable, waste deliverable, and potentially put an increased strain on the liver.

The ratio of the deliverable to the emulsion system is preferably from 1:4-8 by weight, with deviations up to 20% by weight being included, and with deviations up to 10% being more preferred, thus 1:4-8±20% by weight or 1:4-8±10% more preferred by weight. The relatively large amount of deliverable solubilized in the described emulsion systems in relation to conventional systems, where an approximately 2-3% solubilized deliverable is expected, is a significant and unexpected improvement that may be attributable to the believed layered structures of the formed microemulsion droplets.

Another believed benefit from the oil-based solution is the ability to form water-core liposomes in addition to the monolayer surfactant bound particles that form the microemulsion in the GI tract. Thus, the previously described zinc and other salts are believed delivered by in situ formed liposomes. The ability of the emulsion system to form water-core liposomes in addition to the surfactant bound particle microemulsion in the GI tract is believed to provide the ability to concurrently deliver alcohol-soluble species, oil-soluble species, and water-soluble deliverables to the tissue of the GI tract. The ability of the oil-based solution to provide such a diverse group of different deliverables with significantly enhanced bioavailability to the bloodstream of a mammal with a single capsule is another significant and unexpected benefit.

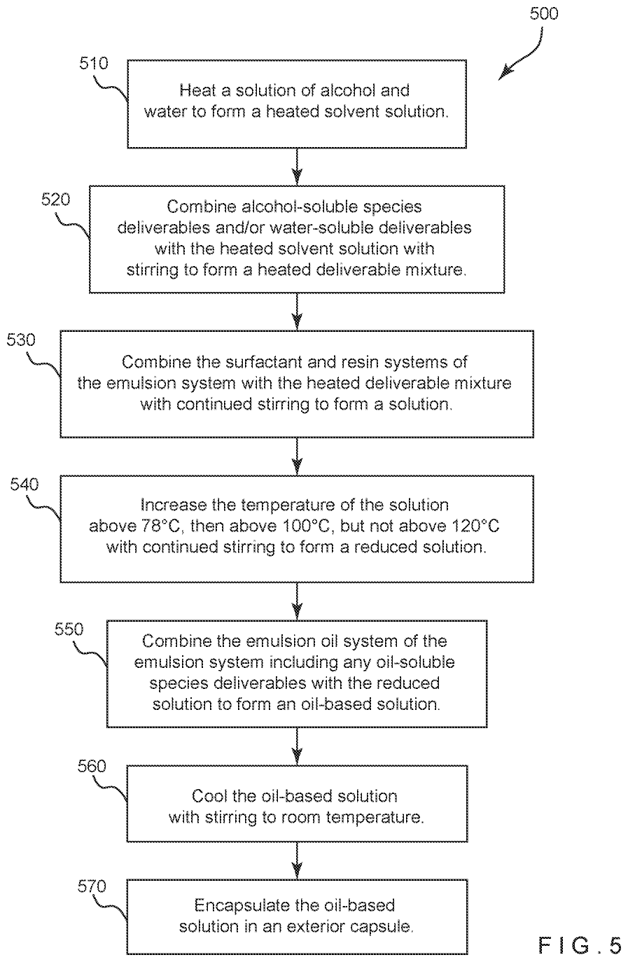

FIG. 5 represents a method 500 of making the composition including the encapsulated oil-based solution. The oil-based solution may optionally include water-soluble deliverables.

In 510, a solvent solution of alcohol and water is heated to form a heated solvent solution 512. The heated solvent solution 512 preferably includes ethanol and water. However, other alcohols may be used that are compatible with later OIW microemulsion formation in the GI tract and the desired deliverables. The ratio of alcohol to water in the heated solvent solution 512 is preferably from 80:20 to 97:3 on a volume basis, more preferably from 90:10 to 95:5 on a volume basis. The heated solvent solution 512 is preferably heated to a temperature from 65° C. to 78° C., more preferably from 68° C. to 75° C.

In 520, alcohol-soluble species deliverables and/or optional water-soluble deliverables are combined with the heated solvent solution 512 with stirring to form a heated deliverable mixture 522. Depending on the solubility of deliverable, some deliverables will fully dissolve during this addition, while others may not. For example, a deliverable with the solubility characteristics of berberine, is unlikely to fully dissolve at this stage.

In 530, the surfactant and resin systems of the emulsion system are combined with the heated deliverable mixture 522 with continued stirring to form a solution 532.

In 540, the temperature of the solution 532 is increased above 78° C., which is the boiling point of ethanol. Preferably the temperature of the solution 532 is then increased above 100° C., which is the boiling point of water. Preferably, the temperature of the solution 532 does not exceed 120° C. Heating and stirring are continued until the ethanol and water are substantially removed to form a reduced solution 542.

In 550, the emulsion oil system of the emulsion system including any oil-soluble species deliverables is combined with the reduced solution 542 to form an oil-based solution 552.

In 560, the oil-based solution 552 is permitted to cool with stirring to room temperature.

In 570, the oil-based solution 552 is encapsulated with an exterior capsule.

The following examples are provided to illustrate one or more preferred embodiments of the invention. Numerous variations can be made to the following examples that lie within the scope of the invention.

### EXAMPLES

Example 1: Formation of an oil-based solution including oil-soluble species, alcohol-soluble species, and/or water-soluble deliverables that forms an OIW microemulsion when released into the GI tract.

The desired alcohol-species and/or water-soluble deliverables were added to a heated water/alcohol solution to at least partially solubilize the deliverables and form a heated deliverable mixture. The surfactant and resin systems of the emulsion system were then added to form a solution. The temperature of the solution was then increased above 100° C. The emulsion-oil system including any oil-soluble species deliverables was then added to the solution. The solution was then allowed to cool to room temperature. Stirring was used throughout until the solution cooled to room temperature. The resulting oil-based solution was then placed into an exterior capsule to form any orally consumable SMEDS.

Example 2: An oil-based solution including deliverables that forms an OIW microemulsion when released into the GI tract for pain relief.

The general method of Example 1 was used to combine the following: from 2 to 3 percent by weight phospholipid, from 24 to 30 percent by weight polyethylene glycol derivative, from 8 to 13 percent by weight turmeric oleoresin, from 1 to 2.5 percent by weight propolis, from 12 to 18 percent by weight associating oil, from 23 to 31 percent by weight turmeric oil, from 5 to 9 percent by weight beta caryophyllene, from 1.5 to 4 percent by weight cannabis extract (“hemp oil”), from 3 to 4 percent by weight curcumin extract, preferably constituting greater than 90% by weight curcumin, and from 2 to 4 percent by weight Boswellia serrata. The composition optionally may include from 1 to 3 percent by weight piperine.

Example 3: An oil-based solution including deliverables that forms an OIW microemulsion when released into the GI tract for balancing microbial load in a mammal.

The general method of Example 1 was used to combine the following: from 3.2 to 5 percent by weight phospholipid, from 26.3 to 30 percent by weight polyethylene glycol derivative, from 3 to 7 percent by weight turmeric oleoresin, from 2.6 to 4 percent by weight propolis, from 18.2 to 23 percent by weight associating oil, from 11 to 20 percent by weight turmeric oil, from 1 to 5 percent by weight cinnamon oil, from 1 to 5 percent by weight peppermint oil, from 0.2 to 1.3 percent by weight cannabis extracts (“hemp oil”), from 0.3 to 2 percent by weight berberine HCl, from 2 to 5 percent by weight milk thistle extract, from 3 to 7 percent by weight artemisinin, from 0.3 to 2 percent by weight Andrographis, from 2 to 6 percent by weight Boswellia serrata, and from 2 to 4 percent by weight quercetin.

Example 4: An oil-based solution including deliverables that forms an OIW microemulsion when released into the GI tract for controlling inflammation.

The general method of Example 1 was used to combine the following: from 1 to 3 percent by weight phospholipid, from 25 to 34 percent by weight polyethylene glycol derivative, from 6 to 10 percent by weight turmeric oleoresin, from 8 to 13 percent by weight associating oil, from 27 to 35 percent by weight turmeric oil, from 2 to 6 percent by weight cinnamon oil, from 7 to 10 percent by weight spearmint oil, from 2 to 5 percent by weight berberine HCl, from 2 to 5 percent by weight milk thistle extract, from 2 to 5 percent by weight resveratrol, from 2 to 5 percent by weight hesperetin, and from 2 to 5 percent by weight quercetin.

Example 5: An oil-based solution including deliverables that forms an OIW microemulsion when released into the GI tract for supplementing dietary zinc.

The general method of Example 1 was used to combine the following: from 1 to 3 percent by weight phospholipid, from 25 to 34 percent by weight polyethylene glycol derivative, from 7 to 10 percent by weight propolis, from 22 to 30 percent by weight associating oil, from 10 to 15 percent by weight turmeric oil, from 10 to 15 percent by weight spearmint oil, from 3 to 5 percent by weight zinc acetate, from 2 to 5 percent by weight luteolin, from 2 to 5 percent by weight hesperetin, and from 2 to 5 percent by weight quercetin.

### Example 6: Comparative Blood Uptake Rates for Oral Curcumin Delivery Via Capsule

An oil-based solution delivery system generally in accord with Example 2 was compared to a commercially available purported capsulated emulsion delivery system for curcumin delivery and bioavailability. The commercially available product was stated on its label to be a cellulose soft gel capsule including sunflower lecithin and 400 mg of curcuminoid powder. The commercially available product is believed to also include TPGS and turmeric oil. The commercially available product may or may not include additional ingredients.

Analysis of the commercially available product determined that an individual capsule included approximately 1 mL of liquid containing 266 mg of curcumin, 76 mg of demethoxycurcumin, and 38 mg of bisdemethoxycurcumin. Performing the same analysis on the encapsulated oil-based solution capsules established that each hard-capsule shell contained approximately 1 mL of liquid containing 50 mg of curcumin, 10 mg of demethoxycurcumin, and 2 mg of bisdemethoxycurcumin.

To provide the same amount of curcumin for accurate uptake performance and bioavailability comparison the following steps were taken. The liquid contents of one and approximately one half of a second of the commercially available capsules was consumed to provide a dose of approximately 400 mg of curcumin, as each capsule was known to contain approximately 266 mg. Thus, one “dose” of the commercially available product included approximately 400 mg of curcumin and had a total encapsulated volume of approximately 1.5 mL. For the oil-based solution capsules, 8 of the capsules were consumed to provide the approximately 400 mg of curcumin, as each capsule was known to contain approximately 50 mg of curcumin. Thus, one “dose” of the oil-based solution included approximately 400 mg of curcumin and had a total encapsulated volume of approximately 6 mL.

On an empty stomach, a human subject took via mouth one dose of either the commercially available product or the oil-based solution. For each, venous blood samples were collected from the human subject before and after dosing (consumption of capsules) at varying time intervals out to approximately three hours for the oil-based blend and out to approximately eight hours for the commercially available product. Thus, a baseline blood sample was collected prior to consumption of either dose. The collected blood samples were subjected to plasma separation via centrifuge and the resulting plasma samples were stored at −25° C. until analysis. The resulting plasma samples were analyzed for curcumin and its metabolites (curcumin-glucuronide and curcumin-sulfate) using LC/MS/MS.

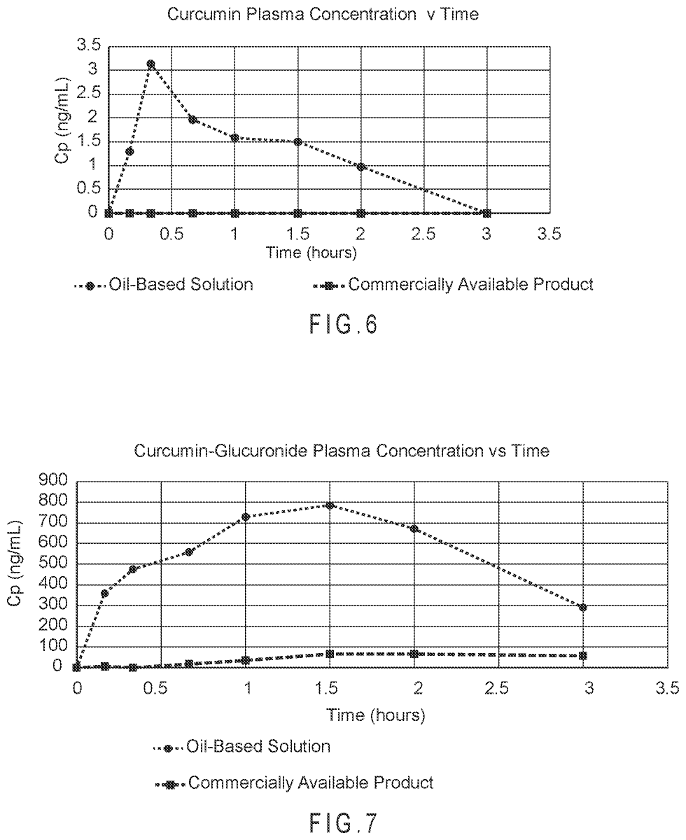

FIG. 6 provides the results from the LC/MS/MS plasma curcumin analysis at the 0, 10, 20, 40, 60, 90, 120, 180, and 480 (commercially available only) minute time intervals. This analysis was directly performed for curcumin, without prior enzyme cleavage of glucuronide. The time after dose consumption by the subject when the blood sample was collected is represented on the X-axis, while the nanograms (ng) of curcumin per milliliter (mL) determined for the plasma samples is represented on the Y-axis.

As can be seen in the figure, the commercially available product provided no measurable plasma free curcumin concentration out to 8 hours. In comparison, the oil-based solution provided a free curcumin plasma maximum concentration of 3.14 ng/mL 20 minutes post consumption and maintained a measurable free curcumin plasma concentration past 2 hours. We believe a therapeutically effective dose of free curcumin is approximately 1 ng/mL, which was maintained by the oil-based solution from approximately 10 minutes until approximately 2 hours after consumption.

It is impossible to compare uptake performance of the oil-based solution with the commercially available product regarding free curcumin bloodstream delivery and bioavailability, as the commercially available product was unable to produce a measurable free curcumin concentration in the plasma. Hence, the commercially available product was incapable of delivering unmetabolized curcumin to the bloodstream of a human subject, while the oil-based solution delivered significant unmetabolized, and thus “free”, curcumin to the bloodstream. A similar result would be expected in other mammals.

FIG. 7 provides the results from the LC/MS/MS plasma curcumin-glucuronide analysis at the 0, 10, 20, 40, 60, 90, 120, 180, and 480 (commercially available only) minute time intervals. This analysis was directly performed for curcumin-glucuronide. The time after dose consumption by the subject when the blood sample was collected is represented on the X-axis, while the nanograms (ng) of curcumin-glucuronide per milliliter (mL) determined for the plasma samples is represented on the Y-axis. Unlike the analysis of FIG. 4, which determined direct bloodstream delivery of free curcumin, the FIG. 5 analysis determined the concentration of the primary metabolite of curcumin generated in the bloodstream in response to dose consumption for the oil-based solution and for the commercially available product.

The oil-based solution provided a peak curcumin-glucuronide concentration of approximately 800 ng/mL approximately 1.5 hours after capsule consumption and maintained a plasma concentration above 300 mg/mL from 10 to 180 minutes after capsule consumption. The commercially available product provided a peak curcumin-glucuronide concentration of approximately 68 ng/mL approximately 1.5 hours after capsule consumption, did not produce a meaningful measured concentration until approximately 40 minutes after capsule consumption, and provided an approximately 60 ng/mL concentration between 3 and 8 hours.

A comparison of metabolized curcumin delivery peak performance shows that the delivery provided by the oil-based solution was approximately twelve times greater, thus more than an order of magnitude greater, than that provided by the commercially available product. From a post-consumption time perspective, the oil-based solution, and the commercially available product, both provided peak bloodstream concentrations of curcumin-glucuronide approximately 1.5 hours after dose consumption. A substantial timing difference between the oil-based solution and the commercially available product was the relatively rapid plasma concentration post-peak decrease of the curcumin-glucuronide provided by the oil-based solution in comparison to the substantially lower, but longer lasting bloodstream curcumin-glucuronide concentration provided by the commercially available product. The constant, low curcumin-glucuronide concentration provided by the commercially available product suggests that the 68 ng/mL plasma concentration for the metabolized curcumin-glucuronide is unlikely to be the byproduct of a therapeutic free curcumin dose.

An Area Under the Curve (AUC) calculation was performed to determine the total curcumin-glucuronide generated from the oil-based solution in relation to the commercially available product. The AUC values provide a measure of the cumulative amount of curcumin-glucuronide in the bloodstream, thus total exposure across a period of time. By cumulative amount it is meant the total bloodstream available curcumin-glucuronide as metabolized from curcumin until the selected time.

The plasma curcumin-glucuronide concentration values for the commercially available product where used as the control (denominator) while the plasma curcumin-glucuronide concentration values for the oil-based solution were used as the numerator to determine the AUC bioavailability values. This was possible as both capsules included approximately 400 mg of curcumin. Thus, the AUC values reflect how many more times of curcumin-glucuronide the oil-based solution generated in the bloodstream at a selected time in relation to the commercially available product.

Table I below provides the calculated results over 3-hours from capsule consumption with estimation due to the slight variability in the withdrawal times of the blood samples.

TABLE I

| | Oil-Based Solution |
|---|---|
| Time (minutes) | Bioavailability Increase |
| 0 | 0 |
| 10 | 50 |
| 20 | 78.3 |
| 40 | 60.7 |
| 60 | 36.3 |
| 90 | 22.3 |
| 120 | 17.1 |
| 180 | 12.7 |

The substantial and rapid generation of metabolized curcumin in the bloodstream by the oil-based solution in relation to the commercially available product is seen during the 10 to 60 minute post-dose time period, with the oil-based solution generating 78 times more metabolized curcumin in the bloodstream after 20-minutes. After 60 minutes, the delivery difference falls, with an average of about 17 times as much being delivered by the oil-based solution in relation to the commercially available product. In combination, these results suggest that the commercially available product fails to form a microemulsion in the GI tract, or at least fails to form a microemulsion that can deliver curcumin to the bloodstream much more effectively than consuming powdered curcumin.

To provide a clear and more consistent understanding of the specification and claims of this application, the following definitions are provided.

An oil-soluble species is a species that is insoluble in water and soluble in medium chain triglyceride (MCT) oils at 50 mg/mL and higher, preferably 100 mg/mL and higher. Oil-soluble species are generally soluble in MCT oils at room temperature and are freely or very soluble in MCT oils at temperatures of 70 degrees Celsius and greater. The term “generally soluble in MCT oils at room temperature” is used because some high purity oil-soluble species are sparingly soluble in MCT oils at room temperature, but are freely or very soluble in the MCT oils above 70 degrees Celsius, and once solubilized in the MCT oils at elevated temperature, will remain solubilized at room temperature. Oil-soluble species neither include nor are water. Thus, liquids and solids may exist that technically are soluble in oil, but because they also are soluble in water or not sufficiently soluble in MCT oils are not “oil-soluble species”.

An alcohol-soluble species is a species that is insoluble in water and has a greater solubility in ethanol than in medium chain triglyceride (MCT) oils. For example, the nonderivatized hormone DHEA is soluble in ethanol up to approximately 150 mg/mL, thus being freely soluble, while having a solubility in MCT oil of only up to approximately 10 mg/mL, thus being only sparingly soluble. Alcohol-soluble species neither include nor are water. Thus, liquids and solids may exist that technically are soluble in alcohol, but because they also are soluble in water or more or equivalently soluble in MCT oils than in ethanol are not “alcohol-soluble species”.

Phosphatidylcholine (PC) molecules are a subset of the larger set of phospholipids and are commonly used to form liposomes in water. When placed in water without other constituents, PC forms liposomes. In the presence of an oil, the application of sufficient shear forces to the PC liposomes in water can produce monolayer structures, including micelles. PC has a head that is water-soluble and a tail that is much less water-soluble in relation to the head. PC is a neutral lipid, but carries an electric dipole moment of about 10 D between the head and the tail, making the molecule itself polar.

Tocopheryl polyethylene glycol succinate 1000 (TPGS) is generally considered a surfactant having a non-polar, oil-soluble “Vitamin E” tail and a polar, water-soluble polyethylene glycol head. TPGS is a member of the polyethylene glycol derivatives that also include polysorbate 20, 40, 60, and 80.

Room temperature and pressure means from 20 to 28 degrees Celsius at approximately 100 kPa.

Solid means a substance that is not a liquid or a gas at room temperature and pressure. A solid substance may have one of a variety of forms, including a monolithic solid, a powder, a gel, a wax, or a paste.

Liquid means a substance that is not a solid or a gas at room temperature and pressure. A liquid is an incompressible substance that flows to take on the shape of its container.

Solutions lack an identifiable interface between the solubilized molecules and the solvent. In solutions, the solubilized molecules are in direct contact with the solvent.

Solubilized means that the deliverable is in the solution of the droplet. When solubilized, dissociation (thus, liquid separation or solid formation) of the deliverable does not result in droplet average particle diameters in excess of 200 nm as determined by DLS and discussed further below, or by the formation of precipitated crystals of the deliverable visible with the naked eye. Thus, if either average particle diameters more than 200 nm or precipitated crystals visible to the naked eye form, the deliverable is not solubilized in the solution of the droplet. If a deliverable is not solubilized in the solution, it is insoluble in the solution. In many respects, solubility may be thought of as a concentration dependent continuum. For example, the following descriptive terms may be used to express solubility of a solute in a solvent (grams solid/mL of solvent) at 25 degrees Celsius:

| Descriptive Level | Parts solvent per 1 part of solute |
|---|---|
| Very Soluble | Less than 1 |
| Freely Soluble | From 1 to 10 |
| Soluble | From 10 to 30 |
| Sparingly Soluble | From 30 to 100 |
| Slightly Soluble | From 100 to 1000 |
| Very Slightly Soluble | From 1000 to 10,000 |
| Insoluble | More than 10,000 |

Dissociation occurs when a previously solubilized solid or liquid leaves a solution and is no longer in direct contact with a solvent of the solution. Dissociation of solids from the solvent occurs through recrystallization, precipitation, and the like. Dissociation of liquids from the solvent occurs through separation and the formation of a visible meniscus between the solvent and the dissociated liquid.

Emulsions are mixtures of two or more liquids that do not solubilize. Thus, one of the liquids carries droplets of the second liquid. The droplets of the second liquid may be said to be dispersed in a continuous phase of the first liquid. An interface, separation, or boundary layer exists between the carrier liquid (continuous phase) and the droplets of the second liquid. Emulsions may be macroemulsions, pseudo-emulsions, microemulsions, or nanoemulsions. The primary differences between macroemulsions, microemulsions, and nanoemulsions are the average diameter of the droplets dispersed in the continuous phase and the stability of the emulsion over time. Pseudo-emulsions are differentiated as solids are present in the emulsion.

Droplets or liquid particles are formed by the hydrophobic “oil” phase of a microemulsion and are carried by the hydrophilic continuous phase. The exterior of the droplets is defined by a boundary layer that surrounds the volume of each liquid droplet. The boundary layer of a droplet defines the exterior surface of the droplets forming the dispersed oil phase of the microemulsion. The continuous phase of the microemulsion resides exterior to the boundary layer of the droplets, and thus, carries the droplets.

Microemulsions are thermodynamically stable dispersions of oil in water, with oil being defined as any water-insoluble liquid. Microemulsion are made by simple mixing of the components. Thus, microemulsions spontaneously form and do not require high shear forces. Unlike macroemulsions, microemulsions do not substantially scatter light. The IUPAC definition of a microemulsion is a “dispersion made of water, oil, and surfactant(s) that is an isotropic and thermodynamically stable system with dispersed domain diameter varying approximately from 1 to 100 nm, usually 10 to 50 nm.” Thus, the droplets of a microemulsion are approximately three orders of magnitude smaller than the droplets of a macroemulsion and are thermodynamically stable.

A visually clear microemulsion has an average particle diameter of 200 nm and less and lacks precipitated solid crystals visible to the naked eye.

Continuous phase means the portion of a microemulsion that carries the droplets that include the deliverable. For example, the oil-in-water (OIW) microemulsions (non-polar droplets in polar continuous phase) addressed herein have oil droplets including the deliverable to be delivered carried in a polar, “water” continuous phase. While the words “water” and “oil” are used, the “water” can be any liquid that is more polar than the “oil” (such as a polar oil), and the “oil” can be any liquid that is less polar than the “water. Thus, the terms “polar continuous phase” and “water continuous phase” are synonymous, unless water is specifically being discussed as one of the microemulsion components.

Average droplet diameter is determined by dynamic light scattering, sometimes referred to as photon correlation spectroscopy. The determination is made between 20 and 25 degrees Celsius. One example of an instrument suitable for average droplet diameter determination is a Nicomp 380 ZLS particle sizer as available from Particle Sizing Systems, Port Richey, Fla. DLS can determine the diameter of droplets in a liquid by measuring the intensity of light scattered from the droplets to a detector over time. As the droplets move due to Brownian motion the light scattered from two or more droplets constructively or destructively interferes at the detector. By calculating the autocorrelation function of the light intensity and assuming a droplet distribution, it is possible to determine the sizes of droplets from 1 nm to 5 micrometers (um). The instrument is also capable of measuring the Zeta potential of droplets.

Ingestible means capable of being ingested through the mouth by a living mammal while edible means fit to be eaten, thus in contrast to being unpalatable or poisonous. Edible also means that the composition has less than the permitted amount of viable aerobic microorganisms and meets the American Herbal Products Association (AHPA) guidelines for metals, adulterants, toxins, residual solvents, and pesticides.

Where a range of values is provided, it is understood that each intervening value, to the tenth of the unit of the lower limit unless the context clearly dictates otherwise, between the upper and lower limit of that range, and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both limits, ranges excluding either or both included limits are also included in the invention.

While various aspects of the invention are described, it will be apparent to those of ordinary skill in the art that other aspects and implementations are possible within the scope of the invention. Accordingly, the invention is not to be restricted except by the attached claims and their equivalents.

1. A composition for delivering a deliverable to the gastrointestinal tract, the composition comprising:
an exterior capsule enclosing an oil-based solution, where the oil-based solution comprises an emulsion system and a deliverable,
where the emulsion system comprises a surfactant system, an emulsion oil system, and a resin system, and
where the deliverable is chosen from an oil-soluble species, an alcohol-soluble species, and combinations thereof.

2. The composition of claim 1 configured to form an oil-in-water microemulsion comprising monolayer surfactant bound particles in an aqueous gastrointestinal tract of a mammal.

3. The composition of claim 2, where the oil-in-water microemulsion is thermodynamically stable.

4. The composition of claim 2 further configured to form liposomes in the aqueous gastrointestinal tract of a mammal, where the liposomes are water-core liposomes comprising a water-soluble deliverable.

5. The composition of claim 2, where the monolayer surfactant bound particles are layered structures having an inner oil-based core, an intermediate resinous layer encapsulating the inner core, and an outer surfactant monolayer encapsulating the intermediate layer.

6. The composition of claim 2, where the monolayer surfactant bound particles have an average droplet diameter of 10 to 100 nanometers.

7. The composition of claim 2, where the monolayer surfactant bound particles have an average droplet diameter of 10 to 80 nanometers.

8. The composition of claim 2, where the monolayer surfactant bound particles have an average droplet diameter of 10 to 60 nanometers.

9. The composition of claim 1, where the surfactant system comprises a phospholipid and a polyethylene glycol derivative.

10. The composition of claim 9 where the phospholipid is a glycerophospholipid isolated from lecithin.

11. The composition of claim 10, where the phospholipid is chosen from phosphatidylcholine (PC), phosphatidylethanolamine (PE), phosphatidylinositol (PI), ceramide phosphoryl ethanolamine (Cer-PE), ceramide phosphoryl choline (SPH), and combinations thereof.

12. The composition of claim 10, where the phospholipid is chosen from phosphatidylcholine, phosphatidylethanolamine, and combinations thereof.

13. The composition of claim 10, where the phospholipid is at least 80% by weight phosphatidylcholine.

14. The composition of claim 9, where the polyethylene glycol derivative is chosen from tocopheryl polyethylene glycol succinate 1000, polysorbate 40, polysorbate 60, polysorbate 80, and combinations thereof.

15. The composition of claim 9, where the polyethylene glycol derivative is chosen from tocopheryl polyethylene glycol succinate 1000, polysorbate 40, and combinations thereof.

16. The composition of claim 1, where the surfactant system comprises from 27% to 35% by weight of the oil-based solution.

17. The composition of claim 9, where a ratio of the phospholipid to the polyethylene glycol derivative is from 1:5 to 1:30 in the oil-based solution.

18. The composition of claim 1 where the emulsion oil system comprises an associating oil chosen from a medium chain triglyceride oil, a citrus oil, and combinations thereof.

19. The composition of claim 18, where the medium chain triglyceride is chosen from caproic acid (hexanoic acid), caprylic acid (octanoic acid), capric acid (decanoic acid), lauric acid (dodecanoic acid), and combinations thereof.

20. The composition of claim 18, where the medium chain triglyceride chosen from caprylic acid, capric acid, and combinations thereof.

21. The composition of claim 18, where the citrus oil is chosen from orange oil, lemon oil, and combinations thereof.

22. The composition of claim 18, where the emulsion oil system further comprises a terpene oil.

23. The composition of claim 22, where the terpene oil is chosen from turmeric oil, cinnamon oil, peppermint oil, spearmint oil, and blends of these terpene oils.

24. The composition of claim 22, where the terpene oil is turmeric oil.

25. The composition of claim 1, where the emulsion oil system comprises from 38% to 55% by weight of the oil-based solution.

26. The composition of claim 1 where the emulsion oil system comprises a ratio of associating oil to terpene oil from 1:1.7 to 1:5.5 in the oil-based solution.

27. The composition of claim 1, where the resin system comprises a resin chosen from turmeric oleoresins, propolis, astaxanthin oleoresin, pine resin, ginger oleoresin, and combinations thereof.

28. The composition of claim 1, where the resin system consists essentially of turmeric oleoresin.

29. The composition of claim 1, where the resin system consists essentially of propolis.

30. The composition of claim 1, where the resin system consists essentially of turmeric oleoresin and propolis.

31. The composition of claim 1, where the resin system comprises from 3% to 18% by weight of the oil-based solution.

32. The composition of claim 30, where a ratio of the propolis to the turmeric oleoresin is from 1:1.7 to 1:5 in the oil-based solution.

33. The composition of claim 1, where a ratio of the resin system to the surfactant system to the emulsion oil system is 1:2-4:3.5-6±20% by weight in the oil-based solution.

34. The composition of claim 1, where the deliverable is chosen from curcumin, Boswellia serrata, quercetin, berberine HCl, milk thistle extract, artemisinin, andrographis, luteolin, resveratrol, diindolylmethane, hesperetin, beta caryophyllene, cannabis extracts, and combinations thereof.

35. The composition of claim 1, where the alcohol-soluble species deliverable is chosen from curcumin, Boswellia serrata, quercetin, berberine HCl, milk thistle extract, artemisinin, andrographis, luteolin, resveratrol, diindolylmethane, hesperetin, and combinations thereof.

36. The composition of claim 1, where the oil-soluble species deliverable is chosen from beta caryophyllene, cannabis extracts, and combinations thereof.

37. The composition of claim 34, where the deliverable further comprises a water-soluble deliverable that is soluble in the oil-based solution.

38. The composition of claim 37, where the water-soluble deliverable is a mineral salt.

39. The composition of claim 37, where the water-soluble deliverable is chosen from a zinc salt, a magnesium salt, a calcium salt, and combinations thereof.

40. The composition of claim 1, where the oil-based solution is configured to solubilize from 50 mg to 200 mg of the deliverable per gram of the oil-based solution.

41. The composition of claim 1, where the oil-based solution comprises 10% to 20% of the deliverable by weight.

42. The composition of claim 1, where the ratio of the deliverable to the emulsion is from 1:4-8±20% by weight.

43. The composition of claim 1, where the oil-based solution is configured to deliver the deliverable chosen from the alcohol-soluble species, the oil-soluble species, and the combinations thereof, through the gastrointestinal tract of a mammal and provide a measurable plasma concentration of the deliverable in a non-metabolized form within 20 minutes of the mammal orally-consuming the composition on an empty stomach.

44. An ingestible and edible composition for pain relief, the composition comprising: an encapsulated oil-based solution including from 2 to 3 percent by weight phospholipid, from 24 to 30 percent by weight polyethylene glycol derivative, from 8 to 13 percent by weight turmeric oleoresin, from 1 to 2.5 percent by weight propolis, from 12 to 18 percent by weight emulsion oil, from 23 to 31 percent by weight turmeric oil, from 5 to 9 percent by weight beta caryophyllene, from 1.5 to 4 percent by weight hemp oil, from 1 to 3 percent by weight piperine, from 4 to 4 percent by weight curcumin, and from 2 to 4 percent by weight Boswellia serrata.

45. An ingestible and edible composition for balancing microbial load in a mammal, the composition comprising: an encapsulated oil-based solution including from 3.2 to 5 percent by weight phospholipid, from 26.3 to 30 percent by weight polyethylene glycol derivative, from 3 to 7 percent by weight turmeric oleoresin, from 2.6 to 4 percent by weight propolis, from 18.2 to 23 percent by weight emulsion oil, from 11 to 20 percent by weight turmeric oil, from 1 to 5 percent by weight cinnamon oil, from 1 to 5 percent by weight peppermint oil, from 0.2 to 1.3 percent by weight hemp oil, from 0.3 to 2 percent by weight berberine HCl, from 2 to 5 percent by weight milk thistle extract, from 3 to 7 percent by weight artemisinin, from 0.3 to 2 percent by weight andrographis, from 2 to 6 percent by weight Boswellia serrata, and from 2 to 4 percent by weight quercetin.

46. An ingestible and edible composition for controlling inflammation, the composition comprising: an encapsulated oil-based solution including from 1 to 3 percent by weight phospholipid, from 25 to 34 percent by weight polyethylene glycol derivative, from 6 to 10 percent by weight turmeric oleoresin, from 8 to 13 percent by weight associating oil, from 27 to 35 percent by weight turmeric oil, from 2 to 6 percent by weight cinnamon oil, from 7 to 10 percent by weight spearmint oil, from 2 to 5 percent by weight berberine HCl, from 2 to 5 percent by weight milk thistle extract, from 2 to 5 percent by weight resveratrol, from 2 to 5 percent by weight hesperetin, and from 2 to 5 percent by weight quercetin.

47. An ingestible and edible composition for supplementing dietary zinc in a mammal, the composition comprising: an encapsulated oil-based solution including from 1 to 3 percent by weight phospholipid, from 25 to 34 percent by weight polyethylene glycol derivative, from 7 to 10 percent by weight propolis, from 22 to 30 percent by weight associating oil, from 10 to 15 percent by weight turmeric oil, from 10 to 15 percent by weight spearmint oil, from 3 to 5 percent by weight zinc acetate, from 2 to 5 percent by weight luteolin, from 2 to 5 percent by weight hesperetin, and from 2 to 5 percent by weight quercetin.

48. A method of forming an oil-based solution for delivering a deliverable to the gastrointestinal tract, the method comprising:
heating an alcohol and water solution to a low temperature of 65° C. to 78° C., where the alcohol and water solution has an alcohol to water ratio from 80:20 to 97:3 on a volume basis to form a heated solvent solution;
combining an alcohol-soluble species deliverable with the heated solvent solution to form a heated deliverable mixture;
combining a surfactant system and a resin system with the heated deliverable mixture;
increasing the heated deliverable mixture above 78° C. to form a reduced solution; and
combining an emulsion oil system with the reduced solution to form an oil-based solution.

49.-83. (canceled)
